(19) United States
(12) Patent Application Publication
(10) Pub. No.: US 2021/0068795 A1
(43) Pub. Date: Mar. 11, 2021

(54) COMPUTER-CONTROLLED REMOTE POWER MODULE

(71) Applicant: Smith & Nephew, Inc., Memphis, TN (US)

(72) Inventors: Constantinos NIKOU, Monroeville, PA (US); Branislav JARAMAZ, Pittsburgh, PA (US); Cedric CORPA DE LA FUENTE, Gibsonia, PA (US)

(21) Appl. No.: 16/642,294

(22) PCT Filed: Aug. 30, 2018
(86) PCT No.: PCT/US2018/048819

§ 371 Date: Feb. 26, 2020

Related U.S. Application Data
(60) Provisional application No. 62/552,606, filed on Aug. 31, 2017.

(51) Int. Cl. A61B 17/00; A61B 90/98; A61B 34/20; A61B 17/16; A61B 17/14

(52) U.S. Cl. CPC A61B 17/00; A61B 90/98; A61B 34/20; A61B 17/1628; A61B 2034/2048; A61B 2017/00734; A61B 2034/2055; A61B 2017/00212; A61B 2017/00221; A61B 17/142

(57) ABSTRACT

A computer-controlled remote power module is disclosed. The remote power module may include an enclosure containing a power converter, a voltage controller, an antenna and a microprocessor. The voltage controller may be in electrical communication with the power converter. The microprocessor may be in electrical communication with the power converter, the voltage controller, and the antenna. The antenna may be configured to receive wireless transmissions from a remote computer control system, and provide a signal to the microprocessor based on the received transmissions. The microprocessor may receive the signal, and, based on the signal, selectively cause the voltage controller to provide power. The remote power module may be configured to selectively provide power from a battery to a tool, such as a surgical tool.

### CLAIM OF PRIORITY

This patent application claims the benefit of priority of U.S. Provisional Patent Application Ser. No. 62/552,606, titled “Computer-Controlled Remote Power Module,” filed on Aug. 31, 2017, which is hereby incorporated by reference herein in its entirety.

### TECHNICAL FIELD

The present disclosure generally relates to battery-controlled surgical tools. More specifically, the present disclosure relates to providing a remote power module for use with battery-controlled surgical tools that is configured to receive control signals from a control system.

### BACKGROUND

The use of computers, robotics, and imaging to provide aid during surgery is known in the art. There has been a great deal of study and development of computer-aided navigation and robotic systems used to guide surgical procedures. For example, a precision freehand sculptor employs a robotic surgery system to assist the surgeon in accurately cutting a bone into a desired shape. In procedures such as total hip replacement (THR), computer-aided surgery techniques have been used to improve the accuracy and reliability of the surgery. Orthopedic surgery guided by images also has been found useful in preplanning and guiding the correct anatomical position of displaced bone fragments in fractures along a good fixation by osteosynthesis.

In a typical arthroscopic procedure, a practitioner may use a navigation system, such as an optical or electromagnetic tracking system, for additional guidance so that any cuts or bone shape alterations to be made are consistent with a surgical plan. Both types of tracking systems involve the attachment of sensors to both the bone to be resected and the cutting instrument to be used by the surgeon.

In most systems, cutting tools such as handheld rotary cutting tools used by a surgeon to prepare a bone surface for implantation of a prosthetic joint component use a wired connection to a control system integrated with, for example, the navigation system to form a computer-aided robotic surgery system. Electrical power and communication signals from the control system are delivered to the cutting tool through the wires. In such an arrangement, the control system can drive the motors on the cutting tool, receive status information from the motors on the cutting tool, and receive position information from sensors within the cutting tool.

There is an increasing desire for robotically controlled tools, like a handheld rotary cutting tool, a drill, or an oscillating cutting tool, to be maximally ergonomic. The communication cables used in a typical wired system can be heavy and interfere with use of the handheld rotary tool. However, transitioning to battery-powered tools creates a new set of issues as existing battery-powered tools typically lack communication and remote control modules and are expensive to replace or modify to work with, and take advantage of, modern computer-aided robotic surgery systems.

### SUMMARY

This summary is submitted with the understanding that it will not be used to interpret or limit the scope or meaning of the present disclosure.

There is also provided a surgical tool system comprising a remote computer control system, and a remote power module configured to be releasably attached to a surgical tool on a first side, to be releasably attached to a battery module on a second side, to wirelessly communicate with the remote computer control system, and to control the provision of power from the battery to the surgical tool. According to certain embodiments, the remote power module comprises an enclosure, a power converter contained within the enclosure, a voltage controller in electrical communication with the power converter and contained within the enclosure, an antenna configured to receive wireless transmissions from the remote computer control system, and a microprocessor in electrical communication with the power converter, the voltage controller, and the antenna. The microprocessor is configured to receive a signal from the antenna, and, based on the signal, selectively cause the voltage controller to provide power to the surgical tool.

According to certain embodiments, the enclosure comprises one or more of polypropylene, polypropylene copolymer, polymethylpentene, polytetrafluoroethylene resin, polymethyl methacrylate, ethylene tetrafluoroethylene, ethylene chlorotrifluoroethlyene, fluoro ethylene propylene, polyether imide, perfluoroalkoxy, polyketone, polyphenylene oxide, polysulfone, polyvinyl chloride, polyvinylidene fluoride, silicone, and thermoplastic elastomers.

According to certain embodiments, the power converter is configured to receive electrical power from the battery module at a first voltage, to provide electrical power to the voltage controller at a second voltage, and to provide electrical power to the microprocessor at a third voltage.

According to certain embodiments, the voltage controller comprises one or more power inputs, one or more power outputs, a control signal input, and a switch configured to selectively connect each of the one or more power inputs to a corresponding one of the one or more power outputs based on a value of the control signal input.

According to certain embodiments, the antenna is positioned on the outside of the enclosure, within the enclosure, or is integrated into the enclosure.

According to certain embodiments, the antenna is further configured to transmit one or more signals to the remote computer controller, and the microprocessor is further configured to establish a wireless communication connection with the remote computer controller through the antenna.

According to certain embodiments, the surgical tool system further comprises a wired optical tracking emitter in electrical communication with the microprocessor. According to certain embodiments, the wired optical tracking emitter comprises a light emitting diode (LED) array.

According to certain embodiments, the surgical tool system further comprises a radio frequency identification (RFID) reader configured to read an RFID tag.

There is provided a remote power module comprising: an enclosure, a power converter contained within the enclosure, a voltage controller in electrical communication with the power converter and contained within the enclosure, an antenna configured to receive wireless transmissions from a remote computer control system, and a microprocessor in electrical communication with the power converter, the voltage controller, and the antenna. The microprocessor is configured to receive a signal from the antenna, and, based on the signal, selectively cause the voltage controller to provide power.

According to certain embodiments, the enclosure comprises one or more of polypropylene, polypropylene copolymer, polymethylpentene, polytetrafluoroethylene resin, polymethyl methacrylate, ethylene tetrafluoroethylene, ethylene chlorotrifluoroethlyene, fluoro ethylene propylene, polyether imide, perfluoroalkoxy, polyketone, polyphenylene oxide, polysulfone, polyvinyl chloride, polyvinylidene fluoride, silicone, and thermoplastic elastomers.

According to certain embodiments, the enclosure is configured to be releasably attached to a battery on a first side and releasably attached to a surgical tool on a second side. According to such embodiments, the power converter is configured to receive electrical power from the battery at a first voltage, to provide electrical power to the voltage controller at a second voltage, and to provide electrical power to the microprocessor at a third voltage.

According to certain embodiments, the voltage controller comprises one or more power inputs, one or more power outputs, a control signal input, and a switch configured to selectively connect each of the one or more power inputs to a corresponding one of the one or more power outputs based on a value of the control signal input.

According to certain embodiments, the antenna is positioned on the outside of the enclosure, within the enclosure, or is integrated into the enclosure.

According to certain embodiments, the antenna is further configured to transmit one or more signals to the remote computer controller; and the microprocessor is further configured to establish a wireless communication connection with the remote computer controller through the antenna.

According to certain embodiments, the remote power module further comprises a wired optical tracking emitter in electrical communication with the microprocessor. According to certain embodiments, the wired optical tracking emitter comprises a light emitting diode (LED) array.

According to certain embodiments, the remote power module further comprises a radio frequency identification (RFID) reader configured to read an RFID tag.

### BRIEF DESCRIPTION OF THE DRAWINGS

The accompanying drawings, which are incorporated in and form a part of the specification, illustrate the embodiments of the present disclosure and together with the written description serve to explain the principles, characteristics, and features of the present disclosure. In the drawings:

FIG. 1 is an illustration of an operating room with a computer-aided robotic surgery system in accordance with an embodiment.

FIG. 2 depicts a sample circuit diagram for a battery-operated tool in accordance with an embodiment.

FIG. 3 depicts a sample diagram of a battery-operated tool including a remote power module in accordance with an embodiment.

FIG. 4 depicts a sample diagram for a remote power module in accordance with an embodiment.

FIG. 5 depicts an alternate sample diagram for a remote power module in accordance with an embodiment.

### DETAILED DESCRIPTION

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

The embodiments of the present teachings described below are not intended to be exhaustive or to limit the teachings to the precise forms disclosed in the following detailed description. Rather, the embodiments are chosen and described so that others skilled in the art may appreciate and understand the principles and practices of the present teachings.

This disclosure describes a remote power module that attaches between a standard battery-operated surgical tool and the battery to provide enhanced control and power modulation. The remote power module can be configured to connect to the battery-operated surgical tool in a similar way as the battery (e.g., a snap lock). The battery can then connect to the remote power module using a similar connection method. The remote power module can include communication and power modulation/switching electronics configured to support a wireless communications link with, for example, a remote control system integrated into a computer-aided robotic surgery system. Thus, the remote control system can provide control signals to the module for controlling the battery-operated tool.

In certain implementations, such a remote power module can be used to implement remote control functionality into a standard battery-operated surgical tool. This could allow, for example, a standard surgical drill to be disabled according to information received by a navigation system integrated into the computer-aided robotic surgery system. In certain implementations, if the navigation system, while tracking the drill using external tracking hardware, determines that the position or trajectory of the drill is incorrect relative to a registered surgical plan (registered with the computer-aided robotic surgery system), the control system can disable the drill so that the surgeon does not inadvertently drill in a position or at a trajectory that is not in accordance with the registered surgical plan.

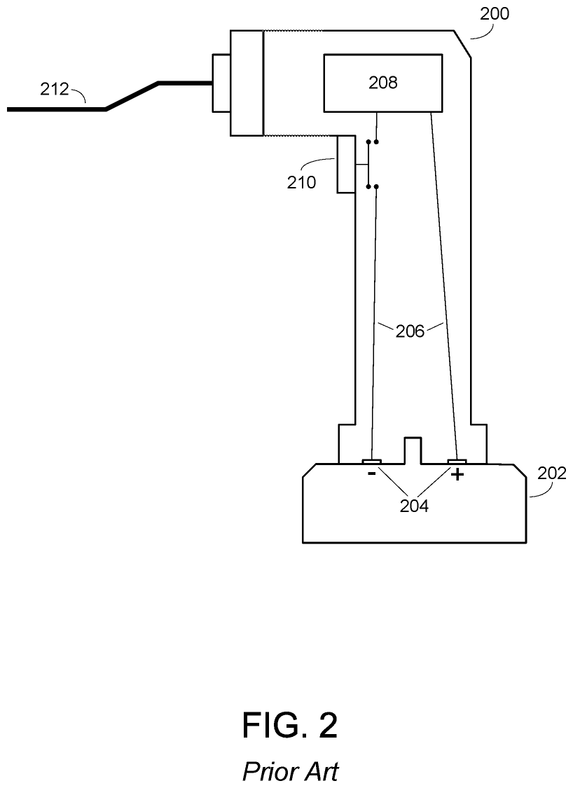

Such an arrangement including a standard battery-operated surgical tool and a remote power module has several advantages. The battery remains standard, and can be removed and charged using existing chargers and charging techniques. A new battery can be swapped in, and, assuming the remote power module is still operably connected to the battery-operated surgical tool, the surgery can continue as before with the enhanced control of the surgical tool. Additionally, battery-operated tools that are designed for use in a surgical environment are typically designed to be completely sealed and more impact resistant to accommodate repeated cleaning and sterilization cycles. Thus, with the remote power module as described herein, the battery-operated surgical tools can remain rugged while maintaining their simplified designs (described in more detail below in regard to FIG. 2), because the various control and communication circuitry is integrated into the remote power module. With such an arrangement, damage to the control and communication circuitry results in replacement of the remote power module rather than the entire tool.

As noted above, a computer-aided robotic surgery system can include various components such as a remote control system and a navigation system. In certain embodiments, the remote control system can include one or more processing devices, firmware-controlled microcontrollers, power systems, communication systems, storage mediums, and other related components. In some examples, the navigation system can be operably connected to the remote control system and be particularly adapted for surgical procedures that utilize tracking devices, such as the NAVIO® surgical navigation system. NAVIO is a registered trademark of BLUE BELT TECHNOLOGIES, INC. of Pittsburgh, Pa.

In alternative embodiments, the disclosure describes a remote power module that attaches between a standard battery-operated tool and the battery to provide enhanced control and power modulation. The remote power module can be configured to connect to the battery-operated tool in a similar way as the battery (e.g., a snap lock). The battery can then connect to the remote power module using a similar connection method. The remote power module can include communication and power modulation/switching electronics configured to support a wireless communications link with, for example, a remote control system integrated into a smart phone or tablet device. Thus, the remote control system can provide control signals to the module for controlling the battery-operated tool.

In alternate implementations, the remote power module can be used to implement remote control functionality into any battery-operated tool, such as a power drill, power driver, power impactor, or reciprocating saw. Similar to previously described embodiments, battery-operated hand tools could be controlled with information received from an optical navigation system that tracks the tool and provides feedback on its position or trajectory. Similarly, the remote control unit could contain an embedded orientation sensing electronics, including one or a combination of accelerometers, magnetometers, gyroscopes, or inertial measurement units. Batter power provided to the tool can be modulated on the tool's orientation relative to gravity. For example, in an embodiment in which the tool is a power drill, the control system can disable the drill if the orientation of the tool is not normal to the direction of the acceleration of gravity.

An orientation control scheme can be developed that considers the tool target orientation, which can also be provided by one or a combination of accelerometers, magnetometers, gyroscopes, or inertial measurement units. The tool target orientation sensing instrumentation can be a standalone unit or contained in a consumer electronic device, such as a smart phone or tablet. Battery power provided to the tool can be modulated based on the tool's orientation relative to the target orientation. For example, if the tool is a power drill and the target is an angled surface, the control system can disable the drill if its orientation is not perpendicular to the angled surface.

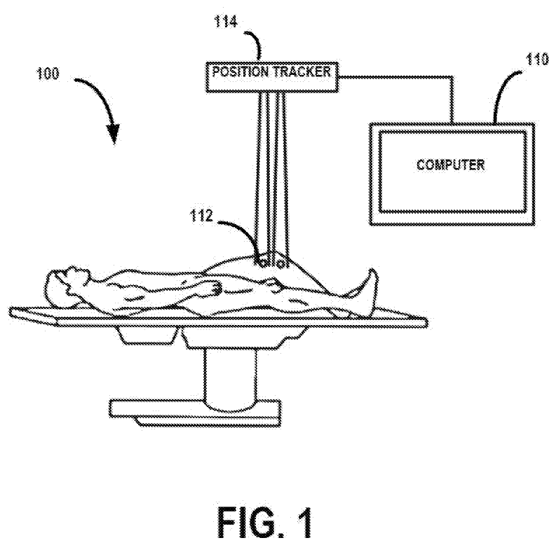

FIG. 1 illustrates components of a computer-aided robotic surgery system 100 that can be configured to perform knee movement tracking according to some embodiments. The computer-aided robotic surgery system 100 can assist a surgeon in performing certain surgical procedures, such as joint revision surgery.

The computer-aided robotic surgery system 100 can include a computer system 110 to provide a display for viewing location data provided by optical trackers 112 as read by a position tracker 114. The optical trackers 112 and position tracker 114 can provide data relevant to the precise location of bones in a knee joint. In certain embodiments, the position tracker 114 can be an optical camera that can detect tracking spheres located on the optical trackers 112 in order to gather location data for a patient upon which a procedure is to be performed. The position tracker 114 can be any suitable tracking system, such as those known in the art to use active trackers, passive trackers, optical trackers, electromagnetic trackers, infrared camera systems, or other similar systems.

Additionally, as noted above, the computer system 110 can be configured to provide communication and control signals to a surgical tool, as well as receive information related to the position/orientation of the surgical tool from the optical trackers or from the surgical tool itself. In certain implementations, the computer-aided robotic surgery system 100 can include additional computing systems. For example, the computer-aided robotic surgery system 100 can include a first computing system configured to compute navigation information, such as patient position information and surgical tool information, and a second computing system configured to compute remote control information related to operation of one or more surgical tools according to a registered surgical plan.

FIG. 2 illustrates a sample battery-powered surgical tool 200. In this example, the surgical tool 200 is an oscillating saw. However, it should be noted that an oscillating saw is shown by way of example only, and any battery-powered surgical tool can be used with the techniques described herein. As examples, the battery-powered surgical tool can include a drill or a rotary cutting tool.

Referring again to FIG. 2, when in operation, or when ready for operation, the surgical tool 200 can be operably connected to battery 202. The battery 202 and the surgical tool 200 can be designed and manufactured to securely connect using a releasable mechanism such as a snap fit or a friction fit. However, because the surgical tool 200 and battery 202 are configured and designed to be used in clean environments such as operating rooms, the surgical tool 200 and the battery are designed to be cleaned and sterilized after each use. Examples of such surgical tools can be found in U.S. Pat. No. 5,263,972 entitled “Surgical Handpiece Chuck and Blade,” the content of which is incorporated herein by reference.

As shown in FIG. 2, the battery 202 can include positive and negative terminals 204. When the battery 202 is attached to the surgical tool 200, the terminals 204 can electrically connect to internal wiring 206 within the surgical tool 200. In certain implementations, the wiring 206 can terminate in exposed copper plates, or plates made from another similar conductive metal, that are positioned such that, when the battery 202 is attached to the surgical tool 200, the terminals 204 abut the plates, thereby electrically connecting the stored electrical energy contained within the battery 202 to the wiring 206. The wiring 206 can be configured such that it establishes an electrical connection between mechanical components 208 within the surgical tool 200 with the battery 202. In certain implementations, the mechanical components 208 can include an electric motor configured to produce a rotational motion from the electrical energy contained within the battery 202. With an oscillating saw as shown in FIG. 2, the mechanical components 208 can also include a drive mechanism that is configured to convert the rotation motion of the motor to an oscillating motion for driving a saw blade 212.

The surgical tool 200 also can include a switch or button 210. The button 210 can be operably connected to an electrical connector configured to short a gap in the wiring 206, thereby activating one or more electrical components contained within the surgical tool 200 (e.g., the electric motor as described above).

In certain implementations, the surgical tool 200 can include additional electrical components such as an external light that can be operated by actuation of button 210 or via an alternate actuation mechanism such as a separate switch (not shown in FIG. 2).

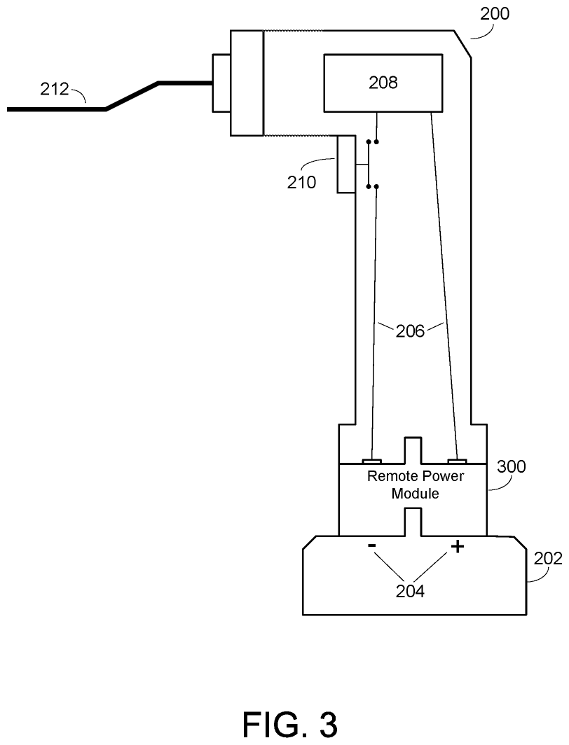

FIG. 3 illustrates the surgical tool 200 of FIG. 2, with an included remote power module 300. As shown in FIG. 3, the remote power module 300 can be positioned between the battery 202 and the surgical tool 200. The remote power module 300 can be designed such that it fastens to the surgical tool 200 in the same manner as the battery 202 would otherwise connect to the surgical tool 200. Similarly, the remote power module 300 can be designed such that the battery 202 connects to the remote power module 300 in the same manner as the battery 202 would normally connect to the surgical tool 200.

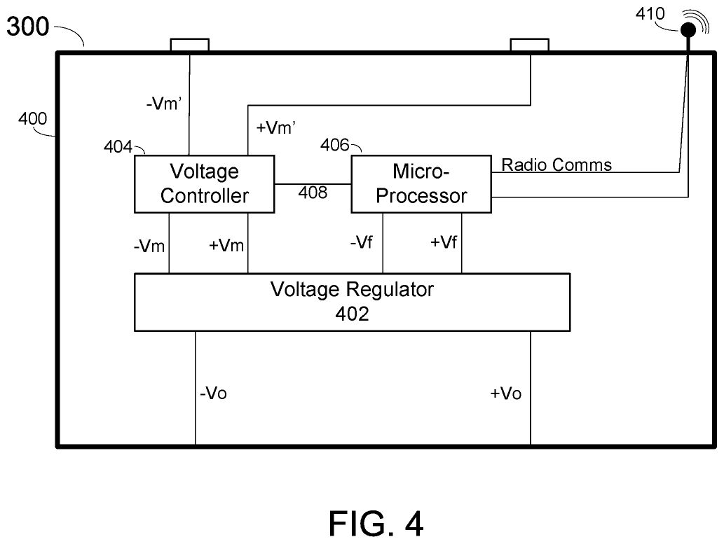

The remote power module 300 can be configured to receive power from the battery 202 via the terminals 204 and provide a control signal to the surgical tool 200 via wiring 206. However, it should be noted that, other than the introduction of the remote power module 300, the design of both the surgical tool 200 and the battery 202 remains unchanged from FIG. 2 to FIG. 3. Thus, through the addition of the remote power module 300, the functionality of the surgical tool 200 can be quickly and easily improved as compared to modifying the internal components of the surgical tool 200 itself. The specific architecture and added functionality of the remote power module is described in the following discussion of FIG. 4.

FIG. 4 illustrates a component view of the remote power module 300 as described in regard to FIG. 3. As noted above, the remote power module 300 can be configured for use in a clean environment such as an operating room. As such, one of ordinary skill in the art would understand the remote power module 300 can be designed and manufactured to be rugged and able to be cleaned/sterilized. To provide for such a design, the various components of the remote power module 300 can be integrated into an enclosure 400. The enclosure 400 can be manufactured from a durable material that is easy to clean and sterilize. For example, the enclosure 400 can be manufactured from a high-strength and durable plastic such as polycarbonate. In other implementations, the enclosure 400 can be manufactured from polymers such as polypropylene (PP), polypropylene copolymer (PPCO), polymethylpentene (PMP), polytetrafluoroethylene (PTFE) resin, polymethyl methacrylate (PMMA or acrylic), ethylene tetrafluoroethylene (ETFE), ethylene chlorotrifluoroethlyene (ECTFE), fluoro ethylene propylene (FEP), polyether imide (PEI), perfluoroalkoxy (PFA), polyketone (PK), polyphenylene oxide (PPO), polysulfone (PSF), polyvinyl chloride (PVC), polyvinylidene fluoride (PVDF), silicone, and thermoplastic elastomers (TPE). In some embodiments, the enclosure can be encapsulated in silicone.

As shown in FIG. 4, the enclosure 400 can be configured to house the various electrical components of the remote power module 300. The remote power module 300 can include a power converter 402, such as a voltage regulator, operably connected to and configured to receive power from a battery (e.g., battery 202 as described above). As shown in FIG. 4, the power converter 402 can be operably connected to the battery and configured to receive power signal +Vo and return power signal −Vo to the battery. In certain implementations, the power converter 402 can be configured to receive power from the battery at a particular voltage (e.g., 18V or 20V).

The power converter 402 can be configured to split the received power, and output the power to additional components. Additionally, the power converter 402 can include one or more feedback control loops and various voltage regulating/modulating components to alter the supplied voltage value as might be needed for additional components. For example, as shown in FIG. 4, the power converter 402 can be configured to provide an input voltage to both a voltage controller 404 (via signal +Vm) as well as to a microprocessor 406 (via signal +Vf). In certain implementations, voltage controller 404 can be configured to act as a switching component configured to selectively provide power to a battery-operated surgical tool (e.g., surgical tool 200 as described above). As such, the power converter 402 can be configured to provide a power signal +Vm to the voltage controller 404 that is the same voltage as the output of the battery (e.g., the same voltage as +Vo). However, in some examples, the microprocessor 406 can operate at a lower voltage than +Vo. In such examples, the power converter 402 can be configured to reduce the input voltage +Vo to the appropriate voltage for operating the microprocessor 406. In certain implementations, the microprocessor 406 can be an ARM (advanced RISC machine) microprocessor configured to implement an ARM instruction set for handling multiple simultaneous processes. Such microprocessors typically operate with a 5V input. In such an implementation, the power converter 402 can be configured to lower the voltage of input signal +Vo to 5V, and output an appropriate 5V input signal +Vf.

As noted above, the voltage controller 404 can be configured to operate as a switch for selectively providing a power signal +Vm′ to operate the surgical tool. The switching mechanism of the voltage controller 404 can be operated via a control signal 408 from the microprocessor 406. For example, the microprocessor 406 can receive instructions from a remote control system that a surgical tool is to be operated under normal circumstances. In such an example, the microprocessor 406 can instruct the voltage controller 404 via signal 408 to close the switch, thereby providing power signal +Vm′ to the surgical tool. The surgical tool can then operate normally, e.g., turn on in response to actuation of a button or similar activation mechanism by a user of the surgical tool. However, if the microprocessor 406 receives instructions from the remote control system that the surgical tool is to stop functioning (e.g., the surgical tool is cutting or drilling outside of an appropriate area), the microprocessor 406 can instruct the voltage controller 404 via signal 408 to open the switch, thereby interrupting power signal +Vm′ to the surgical tool and stopping operation of the surgical tool.

To provide communications between the microprocessor 408 and a remote control system, the remote power module 300 can further include an antenna 410 operably connected to the microprocessor 406. The microprocessor 406 can be configured to send and receive radio communication signals to/from the antenna 410 to communicate with the remote control system. The microprocessor 406 can be designed to establish a wireless communication connection with the remote control system according to a standard communication protocol such as near-field communications (NFC), Bluetooth®, ZigBee, Wi-Fi, or another similar wireless communication standard.

As shown in FIG. 4, the antenna 410 can be positioned on the outside of the remote power module 300. However, this is shown by way of example only. Depending upon the design of the remote power module 300, the position of the antenna 410 can vary accordingly. For example, if the enclosure 400 is made from a material that wireless communication signals can penetrate, the antenna 410 can be positioned within the enclosure 400, thereby reducing the number of openings in the enclosure 400. If the enclosure 400 is made from a material that may interfere with the wireless communication signals, the antenna 410 can be integrated into the enclosure 400 itself. For example, the antenna 410 can be designed as one or more copper traces embedded into the material of the enclosure 400. In some implementations, the microprocessor 406 can include an integrated antenna and communication circuitry, and may not require any additional antennas.

In operation, the components of remote power module 300 can be initiated when the remote power module 300 is operably connected to a battery. Upon initialization, the power converter 402 can generate the appropriate output signals +Vm and +Vf. Similarly, upon initialization, the microprocessor 406 can establish a wireless communication connection with the remote control system and, in response to instructions from the remote control system, signal the voltage controller 404 to either switch into operating mode or into non-operating mode (e.g., either provide power to the surgical tool for standard operation, or interrupt power to the surgical tool). The user of the surgical tool can then use the surgical tool to carry out a registered surgical plan with oversight of the operation of the surgical tool by the remote control system.

In an embodiment, a device may be mounted in or on an autoclavable material, encapsulated in a silicone housing that may be easily sterilized, and removably engagable with a tool. The autoclavable material allows the device to be sterilized or autoclaved a plurality of times without degradation of the material, internal components, or operational performance. For example, the housing may include an internal body or mounting structure (not shown) on components may be mounted. The internal body may be formed of a material that can be subjected to sterilization processes, such as autoclaving. For example, the internal body can be formed from a glass-reinforced epoxy laminate, such as a NEMA grade G-11 glass reinforced epoxy laminate (VETRONITE G11) or equivalent. The internal body may be surrounded by a first covering formed from a first material, such as an over-molding of VMQ silicone material #71385C available from Minnesota Rubber & Plastics, 1100 Xenium Lane N., Minneapolis, Minn. 55441. The housing may also include a second covering that may provide an additional layer of protection or insulation at an outer edge of the housing. The second covering may be formed from a second material, such as an over-molding of VMQ silicone material #71325C available from Minnesota Rubber & Plastics, 1100 Xenium Lane N., Minneapolis, Minn. 55441. The housing may further include a coupling member that passes through the internal body and that engages one or more attachable components. The coupling member may be formed from polysulfone, such as a GEHR PPSU polyphenylsulfone RAL 9005 Black (Solvay Radel R-5500) or equivalent, and can be at least partially covered by the first covering.

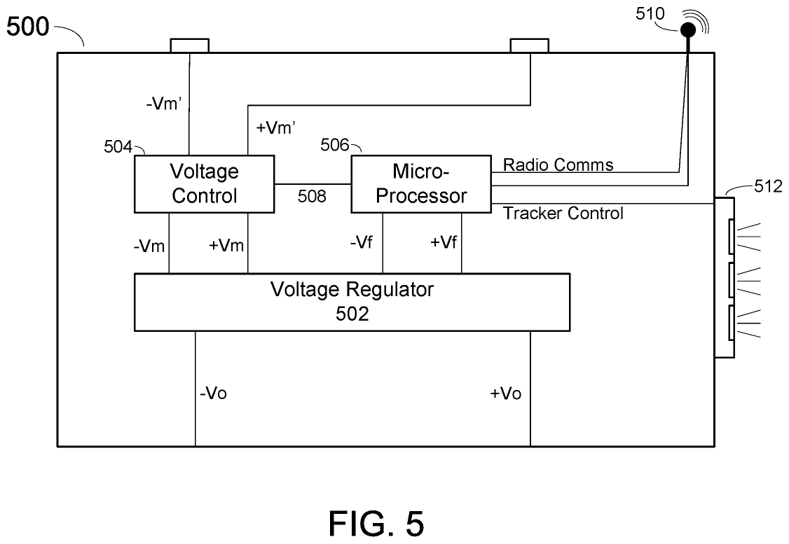

In some implementations, a remote power module can include external tracking hardware in the design of its external enclosure. For example, as shown in FIG. 5, a remote power module 500 can include an array of active LED markers that are detectable by a navigation system such as those described herein. In another embodiment, reflective spheres can be attached to the power module 500.

As shown in FIG. 5, the remote power module 500 includes similar components to remote power module 300 as described above. For example, the remote power module 500 includes a power converter 502 operably connected to a battery (not shown in FIG. 5) and configured to produce signal +Vm for providing power to a voltage controller 504 and signal +Vf for providing power to a microprocessor 506. As described above, microcontroller 506 can be configured to provide a control signal via signal 508 to the voltage controller 504 to either provide or interrupt power to a surgical tool operably connected to the remote power module 500. Additionally, the remote power device 500 can include an antenna 510 operably connected to the microprocessor 506.

In certain implementations, the remote power module 500 can also include a wired optical tracking emitter 512. As shown in FIG. 5, the tracking emitter 512 can be implemented as an LED array including multiple infrared LEDs configured to strobe, blink, or otherwise emit light in a particular pattern upon receiving instructions from the microprocessor 506. A navigation system can then track and monitor the position of the remote power module 500 and, by extension, the surgical tool being used.

For example, in operation, a surgeon or other operator can connect the remote power module 500 to a surgical tool, and then connect a battery to the remote power module 500. Various components such as the power converter 502 and the microprocessor 506 of the remote power module 500 can initiate operation. As noted above, upon initialization, the microprocessor 506 can establish a wireless communication connection to a remote control system. Upon establishing the connection, a navigation system in communication with the remote control system can determine a location and orientation of the remote power module 500 via the tracking emitter 512.

Upon determining a location of the remote power module, the remote control system can prompt the surgeon or other operator to identify what type of surgical tool has been connected to the remote power module 500. Upon receiving a selection of the type of surgical tool connected, the remote control system can load various information about that surgical tool such as dimensional information. Additionally, depending upon what type of surgical tool is being used, the remote control system can prompt for additional information. For example, if a drill is being used, the remote control system can prompt for additional information such as drill bit diameter and length. This information can be used to determine the position of the tip of the drill bit relative to the tracking emitter 512, thereby resulting in the navigation system accurately tracking the drill bit.

It should be noted that prompting for the type of surgical tool being used is provided by way of example only. Additional techniques can be used to identify what type of surgical tool is being used. For example, each surgical tool can have a tag such as an RFID tag. The remote power module 500 can include an RFID reader that is configured to read the tag associated with the surgical tool and identify what type of surgical tool is being used. This information can then be sent by the microprocessor 506 to the remote control system. Alternatively, the microprocessor 506 and/or the voltage controller 504 can be configured to monitor various electrical characteristics of the power being used by the surgical tool such as electrical current draw. The microprocessor 506 and/or the voltage controller 504 can analyze the electrical characteristics to determine what type of surgical tool is being used.

In certain implementations, the surgeon or operator of the surgical tool also can be prompted to perform registration and calibration of the surgical tool. Rather than being prompted to input specific information about the tool (e.g., drill bit length and diameter), the surgeon can be instructed to position the surgical tool in a specific manner. For example, if using a drill, the surgeon can be instructed to position the drill such that the drill bit tip is touching a fiduciary marker whose position is known to the navigation system. Upon touching the fiduciary marker, the navigation system can use the known position of the fiduciary marker in combination with information from the tracking emitter to determine a position and orientation of the surgical tool.

In certain implementations, by including an active tracking emitter such as tracking emitter 512 into a remote power module, an existing surgical tool can be both tracked and controlled without external clamping of tracking hardware onto the surgical tool itself, or modifying the internal components of the surgical tool. Such an arrangement provides for easy modification and improvement of existing tools.

It should be noted that active optical tracking emitters are described by way of example only. In some implementations, a remote power module as described herein can include additional tracking hardware, such as reflective or other similar visual markers, integrated into the design of its external case or enclosure. Such reflective or visual markers can be positioned about the external case of the remote power module such that a navigation system can track the tool associated with the remote power module regardless of the position or orientation that the tool is in.

In some examples, if there is enough power capacity in the battery, additional functionality can be incorporated into the remote power module as described herein. For example, for a handheld cutting device, such as a rotary cutting device, using a bur to cut bone, the remote power module also can be configured to provide motor control signals received from a control system to drive a guard motor, thereby moving the bur guard and exposing the bur for cutting. Similarly, if a navigation system detects that the bur is approaching an area that is not to be removed, the control system can send a command to the remote power module to shut off power to the bur and instruct the guard motor to move the bur guide such that the bur is covered or otherwise unable to continue removing bone.

Additionally, in certain implementations, the remote power module can also receive feedback information from, for example, a motor or motor controller. The feedback can include various information related to the operation of a tool, such as speed and torque information. The speed information can be used by the remote power module to more accurately control the speed of the tool by calibrating its motor control signals. In some examples, torque information can be used as an input to a torque limiting circuit implemented by, for example, an onboard microcontroller in the remote power module that prevents a drill from exerting too much torque on a bone or soft tissue. In another example, torque information can be used as an input to a torque limiting circuit implemented by, for example, an onboard microcontroller in the remote power module that prevents a screw driver from exerting too much torque on a screw or a half-pin.

It should be noted that the remote power module as described herein is described as being configured to be used with a surgical tool by way of example only. The remote power module, and the associated features and functionality, as described herein can be used with any battery-powered tool or device to provide various added benefits and functions.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices also can “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A surgical tool system comprising:
a remote computer control system; and
a remote power module configured to be releasably attached to a surgical tool on a first side, to be releasably attached to a battery module on a second side, to wirelessly communicate with the remote computer control system, and to control the provision of power from the battery to the surgical tool.

2. The surgical tool system of claim 1, wherein the remote power module comprises:
an enclosure;
a power converter contained within the enclosure;
a voltage controller in electrical communication with the power converter and contained within the enclosure;
an antenna configured to receive wireless transmissions from the remote computer control system; and
a microprocessor in electrical communication with the power converter, the voltage controller, and the antenna, wherein the microprocessor is configured to:
receive a signal from the antenna, and
based on the signal, selectively cause the voltage controller to provide power to the surgical tool.

3. The surgical tool system of claim 2, wherein the enclosure comprises one or more of polypropylene, polypropylene copolymer, polymethylpentene, polytetrafluoroethylene resin, polymethyl methacrylate, ethylene tetrafluoroethylene, ethylene chlorotrifluoroethlyene, fluoro ethylene propylene, polyether imide, perfluoroalkoxy, polyketone, polyphenylene oxide, polysulfone, polyvinyl chloride, polyvinylidene fluoride, silicone, and thermoplastic elastomers.

4. The surgical tool system of claim 2, wherein the power converter is configured to receive electrical power from the battery module at a first voltage, to provide electrical power to the voltage controller at a second voltage, and to provide electrical power to the microprocessor at a third voltage.

5. The surgical tool system of claim 2, wherein the voltage controller comprises:
one or more power inputs;
one or more power outputs;
a control signal input; and
a switch configured to selectively connect each of the one or more power inputs to a corresponding one of the one or more power outputs based on a value of the control signal input.

6. The surgical tool system of claim 2, wherein the antenna is positioned on the outside of the enclosure.

7. The surgical tool system of claim 2, wherein the antenna is integrated into the enclosure.

8. The surgical tool system of claim 2, wherein the antenna is positioned within the enclosure.

9. The surgical tool system of claim 2, wherein:
the antenna is further configured to transmit one or more signals to the remote computer controller; and
the microprocessor is further configured to establish a wireless communication connection with the remote computer controller through the antenna.

10. The surgical tool system of claim 2, wherein the remote power module further comprises:
a wired optical tracking emitter in electrical communication with the microprocessor.

11. The surgical tool system of claim 10, wherein the wired optical tracking emitter comprises a light emitting diode (LED) array.

12. The surgical tool system of claim 2, wherein the remote power module further comprises:
a radio frequency identification (RFID) reader configured to read an RFID tag.

13. A remote power module comprising:
an enclosure;
a power converter contained within the enclosure;
a voltage controller in electrical communication with the power converter and contained within the enclosure;
an antenna configured to receive wireless transmissions from a remote computer control system; and
a microprocessor in electrical communication with the power converter, the voltage controller, and the antenna, wherein the microprocessor is configured to:
receive a signal from the antenna, and
based on the signal, selectively cause the voltage controller to provide power.

14. The remote power module of claim 13, wherein the enclosure comprises one or more of polypropylene, polypropylene copolymer, polymethylpentene, polytetrafluoroethylene resin, polymethyl methacrylate, ethylene tetrafluoroethylene, ethylene chlorotrifluoroethlyene, fluoro ethylene propylene, polyether imide, perfluoroalkoxy, polyketone, polyphenylene oxide, polysulfone, polyvinyl chloride, polyvinylidene fluoride, silicone, and thermoplastic elastomers.

15. The remote power module of claim 13, wherein the enclosure is configured to be releasably attached to a battery on a first side and releasably attached to a surgical tool on a second side.

16. The remote power module of claim 15, wherein the power converter is configured to receive electrical power from the battery at a first voltage, to provide electrical power to the voltage controller at a second voltage, and to provide electrical power to the microprocessor at a third voltage.

17. The remote power module of claim 13, wherein the voltage controller comprises:
one or more power inputs;
one or more power outputs;
a control signal input; and
a switch configured to selectively connect each of the one or more power inputs to a corresponding one of the one or more power outputs based on a value of the control signal input.

18. The remote power module of claim 13, wherein the antenna is positioned on the outside of the enclosure.

19. The remote power module of claim 13, wherein the antenna is integrated into the enclosure.

20. The remote power module of claim 13, wherein the antenna is positioned within the enclosure.

21. The remote power module of claim 13, wherein:
the antenna is further configured to transmit one or more signals to the remote computer controller; and
the microprocessor is further configured to establish a wireless communication connection with the remote computer controller through the antenna.

22. The remote power module of claim 13, further comprising:
a wired optical tracking emitter in electrical communication with the microprocessor.

23. The remote power module of claim 22, wherein the wired optical tracking emitter comprises a light emitting diode (LED) array.

24. The remote power module of claim 13, further comprising:
a radio frequency identification (RFID) reader configured to read an RFID tag.
